(19) United States
(12) Patent Application Publication
(10) Pub. No.: US 2025/0268918 A1
(43) Pub. Date: Aug. 28, 2025

(54) METHODS FOR MAKING REDUCED PARTICLE SIZE COMPOUNDS

(71) Applicant: TaP Pharmaceuticals, AG, Baar (CH)

(72) Inventors: Nilesh Parikh, Irvine, CA (US); Kartik Shah, Mumbai (IN)

(73) Assignee: TaP Pharmaceuticals, AG, Baar (CH)

(21) Appl. No.: 18/590,780

(22) Filed: Feb. 28, 2024

(51) Int. Cl. A61K 31/573; A61K 9/16

(52) U.S. Cl. CPC A61K 31/573; A61K 9/1611; A61K 9/1682

(57) ABSTRACT

The invention relates to methods for making a suspension of a size-reduced population of drug particles dispersed in a liquid vehicle, the size-reduced population of drug particles possessing each of the PSD parameters: D10≤0.15 μm, D50≤0.75 μm, and D90≤2.5 μm. In such methods, the drug is a steroid, or a pharmaceutically acceptable salt thereof, the vehicle contains surfactant, salt, and water. The method steps include providing a slurry of a starting population of the drug particles, dispersed in the vehicle and possessing at least one of the PSD parameters: D10>0.15 μm; D50>0.75 μm; and D90>2.5 μm; and subjecting the slurry to bead milling or high-pressure homogenization and concomitantly: reducing in size of the starting population of drug particles to possesses each of the PSD parameters: D10≤0.15 μm, D50≤0.75 μm, and D90≤2.5 μm.

### FIELD

The invention relates to methods for making particles of poorly water-soluble drugs and other compounds that are of reduced size, to a surprising extent, and therefore possesses advantageous properties (e.g., increased bioavailability) while displaying surprising stability. Reduced-particle-sized drugs and other compounds made according to the methods of the present invention unexpectedly lack any appreciable propensity to flocculate or agglomerate due to interparticle attractive forces.

### BACKGROUND

Prednisolone is a steroid hormone, more specifically a corticosteroid, and more specifically a glucocorticosteroid made from hydrocortisone (cortisol). Its structural formula is:

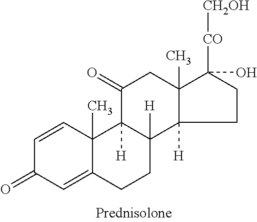

Prednisolone is used in certain pharmaceutical products and is poorly soluble in water. It is used to treat certain types of allergies, inflammatory conditions, autoimmune disorders, and cancers. In the case of ocular inflammatory conditions, prednisolone has been formulated into commercialized eye drop products (e.g. PRED FORTE® and OMNI PRED®). Both of those products comprise suspensions of prednisolone acetate. Both products are indicated for the treatment of steroid-responsive inflammation of palpebral and bulbar conjunctiva, cornea, and anterior segment of the globe.

Loteprednol is another glucocorticosteroid that differs from prednisolone only by having an ester, instead of a ketone, at position 20. Loteprednol is also used to treat ocular inflammatory conditions and has been formulated into commercialized eye drop products (e.g., LOTOMAX® and LOTOMAX SMR). Both products are indicated for the treatment of post-operative inflammation and pain following ocular surgery.\

United State U.S. Pat. No. 10,596,107 (the “'107 patent”) teaches ophthalmic suspensions comprising an ophthalmic active agent (e.g., loteprednol) suspended in a vehicle that comprises carboxyvinyl polymer as suspending agent and a non-ionic cellulose derivative, and in which the ophthalmic active agent is present as particles that have a particle size distribution (“PSD”) of Dᵥ₉₀<5 μm and Dᵥ₅₀<1 μm. (The '107 patent, Col. 2, lines 23-26 and Col. 4, lines 62-64.) The '107 patent discloses that two options for particle size reduction were investigated: a high-pressure homogenizer (Microfluidics model M110-E) and bead milling. In a first set of experiments disclosed by the '107 patent, studies were conducted with the microfluidizer on vehicles composed of 10% loteprednol etabonate, 1% polysorbate 20, 0.5% boric acid, together with a variety of other excipients including tyloxapol, Pluronic F68, benzalkonium chloride (“BAK”), and hydroxypropylmethylcellulose (“HPMC”). The '107 patent states that it was determined from these experiments that polysorbate 20 was not a critical excipient for milling (The '107 patent, Col. 12, lines 1-21.) Table 1 of the '107 patent, closely recreated here as Table A, reports the results of those experiments. As can be seen, no such vehicle subjected to high-pressure homogenization achieved a loteprednol PSD that met the '107 patent's target of Dᵥ₉₀<5 and Dᵥ₅₀<1.

TABLE A
PSD of LE Milling in Polysorbate 20

| Sample Vehicle | Dv10 | Dv50 | Dv90 |
|---|---|---|---|
| 1% tween 20 + 0.1% HPMC in 5% boric | 0.758 | 1.875 | 3.611 |
| 1% tween 20 + 0.5% F68 in 5% boric | 1.248 | 3.332 | 6.419 |
| 1% tween 20 + 0.5% tyloxapol in 5% boric | 0.642 | 1.543 | 3.016 |
| 1% tween 20 + 1% HPMC in 5% boric | 0.593 | 1.257 | 2.344 |
| 1% tween 20 + 0.07% BAK in 5% boric | 0.543 | 1.098 | 2.158 |
| 1% HPMC + 0.2% BAK in 5% boric | 0.472 | 0.940 | 2.068 |

The '107 patent reports results of additional high-pressure homogenization milling trials for Vehicles A-D. Each of which contained 10% loteprednol and 0.5% boric acid. Vehicle A further contained 0.2% BAK and 0.5% HPMC E3. Vehicle B further contained 0.2% BAK and 0.5% poloxamer 407. Vehicle C further contained 0.2% BAK and 0.5% PVP 30. Vehicle D further contained 0.5% CMC LV and 0.2% poloxamer 407. Also, the '107 patent disclosed that Vehicles A-D were milled in the microfluidizer for 20 minutes in a recirculating manner at 25,000 pounds per square inch. The PSD results for Vehicles A-D were reported graphically in its FIG. 1 (not recreated here). The '107 patent made no comment on whether any of Vehicles A-D met the '107 patent's target PSD for loteprednol. (The '107 patent, Col. 12, lines 39-52.)

The '107 patent described yet another set of high-pressure homogenization milling experiments conducted on vehicles that only differed from Vehicle B by having 10%, 20%, or 30% loteprednol. Samples from such vehicles were taken at 10-, 20-, and 30-minute intervals of high-pressure homogenizations. The results were graphically reported in FIG. 2 (not recreated here). (The '107 patent, Col. 12, lines 53-63.)

The '107 patent did not comment on whether any of Vehicles A-D met its target PSD for loteprednol. It did comment that the 30-minute sample with 10% loteprednol had suitable results. (The '107 patent, Col. 12, lines 39-63.)

The '107 patent also graphically reported in its FIG. 4 (not recreated here) the results of high-pressure homogenization milling experiments conducted on Vehicle B, for longer homogenization intervals of 30-, 60-, 90-, 120-, 150-, and 180-minutes. The '107 patent commented that at 90-minutes, a particle size of Dᵥ₉₀ less than 1 μm was achieved. And additional milling time slowly further reduced particle size but did not yield particle sizes achievable by bead milling. (The '107 patent, Col. 13, lines 14-23.) The results of the referenced bead milling experiments were graphically reported in FIG. 3 (not recreated here); and their experimental conditions described in the paragraph bridging Columns 12 and 13. The '107 patent commented that the particle size of both bead-milled samples was smaller than the 30-minute. It then stated that since the described experiments indicated smaller particle size was obtained by bead milling, additional work was done to further optimize the bead milling process. (The '107 patent, Col. 13, lines 14-23 and 24-26.)

As used in the pharmaceutical arts, the term “bioavailability” means the extent to which a drug or other compound becomes available in a tissue targeted for therapy, imaging, etc. by the drug or other compound after its administration. Low bioavailability plagues drugs and other compounds that are poorly soluble in water because of their tendency to be eliminated from the gastrointestinal tract, or the pulmonary or ophthalmic or otic surfaces, prior to being absorbed into the target tissue. Often times, a driver for the low bioavailability of a poorly water-soluble drug or other compound is its slow rate of dissolution in aqueous systems. Additionally, drugs or other compounds that are poorly soluble in water tend to be unsafe for intravenous administration techniques, which are largely reserved drugs or other compounds that are fully water soluble.

It is settled that the dissolution rate of a particulate drug often times increases with increasing surface area (i.e., decreasing particle size). Consequently, methods of making finely divided particulate drugs have been studied and efforts made to control the size and size range of drug particles in pharmaceutical compositions. For example, dry milling techniques have been used to reduce particle size and hence influence drug absorption. However, in conventional dry milling, as discussed by, the limit of fineness is reached in the region of 100 microns (100,000 nm) when material cakes on the milling chamber. (See, e.g., Lachman et al., The Theory and Practice of Industrial Pharmacy, Chapter 2, “Milling”, p. 45, (1986). Lachman et al. also teaches that wet grinding is beneficial in further reducing particle size, but that flocculation restricts the lower particle size limit to approximately 10 microns.

### SUMMARY

Certain embodiments of the present invention provide methods for making a microfine suspension that consists essentially of a size-reduced population of drug particles dispersed in a liquid, aqueous vehicle, the size-reduced population of drug particles possessing each of the PSD parameters: D10≤0.15 μm, D50<0.75 μm, and D90≤2.5 μm. In such methods, the drug is a steroid, or a glucocorticosteroid (e.g., prednisolone), or a pharmaceutically acceptable salt thereof, the vehicle consists essentially of a surfactant, a salt, and water, and the method consists essentially of the following steps. In preferred embodiments, the order of addition vis-à-vis the vehicle components is combining water and salt followed by adding particulate drug and surfactant, in no particular order or simultaneously. Such methods include the further steps of providing a slurry of a starting population of the drug particles, dispersed in the vehicle and possessing at least one of the PSD parameters: D10>0.15 μm; D50>0.75 μm; and D90>2.5 μm, and then subjecting the slurry to bead milling and/or high-pressure homogenization and thereby concomitantly: reducing in size of the starting population of drug particles so that it possesses each of the PSD parameters: D10≤0.15 μm, D50≤0.75 μm, and D90≤2.5 μm, and making the suspension.

In some embodiments, the amount of: particulate drug in the vehicle is from 1% w/v to 30% w/v, surfactant in the vehicle is from 0.125% w/v to 1.5% w/v, and salt in the vehicle is from 0.5% w/v to 25% w/v.

In some embodiments, the drug is a corticosteroid selected from the group consisting of budesonide, betamethasone, cortisone, dexamethasone, hydrocortisone, mometasone, prednisolone, prednisone, and triamcinolone.

In some embodiments, the surfactant is selected from the group consisting of sodium lauryl sulfate, docusate sodium, phosphatidylcholine, lecithin, betaines, tyloxapol, polyoxyethylene sorbitan ester, polyethoxylated castor oil, polyethoxylated hydrogenated castor oils, and poloxamer.

In some embodiments, the salt is selected from the group consisting of calcium acetate, calcium chloride, calcium citrate, calcium diphosphate, calcium maleate, calcium mesylate, calcium nitrate, calcium nitrite, calcium phosphate, calcium sulfate, calcium tartrate, potassium acetate, potassium chloride, potassium citrate, potassium diphosphate, potassium maleate, potassium mesylate, potassium nitrate, potassium nitrite, potassium phosphate, potassium sulfate, potassium tartrate, sodium acetate, sodium chloride, sodium citrate, sodium diphosphate, sodium maleate, sodium mesylate, sodium nitrate, sodium nitrite, sodium phosphate, sodium sulfate, sodium tartrate, and combinations thereof.

In some embodiments, the drug is prednisolone, the surfactant is the at least one of the polyoxyethylene sorbitan ester and the tyloxapol, and the salt is at least one of the sodium citrate and sodium sulfate.

In some embodiments, the slurry is subjected to the bead milling for 12 hours to 60 hours at from 4° C. to 50° C., and wherein the beads are from 1 mm to 10 mm in diameter and of material selected from agate, alumina, ceramic, flint, stainless steel, steel chrome, tungsten carbide, and zirconia, or mixtures thereof.

In some embodiments, the slurry is subjected to the high-pressure homogenization for 15 passes to 250 passes, at from 10,000 PSI to 50,000 PSI and from 4° C. to 50° C.

In some embodiments, the beads are 3 mm in diameter and of the zirconium, and the drug is prednisolone acetate.

In some embodiments, the drug is prednisolone acetate.

Certain embodiments of the present invention provide methods for making a suspension that consists essentially of a size-reduced population of prednisolone acetate particles dispersed in a liquid, aqueous phase, the size-reduced population of prednisolone acetate particles possessing each of the PSD parameters: D10≤0.15 μm, D50<0.75 μm, and D90≤2.5. In such embodiments, the vehicle consists essentially of: from 0.125% w/v to 1.5% w/v of a polysorbate, a tyloxapol, or combinations thereof; from 0.5% w/v to 25% w/v of a salt that is selected from the group consisting of calcium acetate, calcium chloride, calcium citrate, calcium diphosphate, calcium maleate, calcium mesylate, calcium nitrate, calcium nitrite, calcium phosphate, calcium sulfate, calcium tartrate, potassium acetate, potassium chloride, potassium citrate, potassium diphosphate, potassium maleate, potassium mesylate, potassium nitrate, potassium nitrite, potassium phosphate, potassium sulfate, potassium tartrate, sodium acetate, sodium chloride, sodium citrate, sodium diphosphate, sodium maleate, sodium mesylate, sodium nitrate, sodium nitrite, sodium phosphonium sulfate, sodium tartrate, and combinations thereof, and water. In such methods, the amount of prednisolone acetate in the vehicle is from 1% w/v to 30% w/v. And such method consists essentially of the steps of: providing a slurry of a starting population of prednisolone acetate particles, which are dispersed in the vehicle and possess at least one of the PSD parameters: D10>0.1 μm; D50>0.5 μm; and D90>2 μm, and subjecting the slurry to bead milling and/or high-pressure homogenization and thereby concomitantly: reducing the size of the starting population of prednisolone acetate particles so that it possesses each of the PSD parameters: D10≤0.15 μm, D50≤0.75 μm, and D90≤2.5, and D90≤2 μm; and making the suspension.

In some embodiments, the slurry is subjected to the bead milling for from 12 hours to 60 hours at from 4° C. to 50° C., and the beads are from 1 mm to 10 mm in diameter and of a material selected from the group consisting of agate, alumina, ceramic, flint, stainless steel, steel chrome, tungsten carbide, and zirconia, or combinations thereof.

In some embodiments, the beads are 3 mm in diameter and of the zirconium. In some embodiments, the slurry is subjected to the high-pressure homogenization for from 15 passes to 250 passes, at from 10,000 PSI to 100,000 PSI and from 4° C. to 50° C.

### DETAILED DESCRIPTION

The invention provides to methods for making particles of poorly water-soluble drugs and other compounds that are of reduced size, to a surprising extent, and therefore possesses advantageous properties (e.g., increased bioavailability) while displaying surprising stability. Reduced-particle-sized drugs and other compounds made according to the methods of the present invention unexpectedly lack any appreciable propensity to flocculate or agglomerate due to interparticle attractive forces.

Certain embodiments of the present invention provide methods for making suspensions of a size-reduced particulate drug dispersed in a liquid vehicle. The size-reduced particulate drug comprises, or consists essentially of, or consists of, a plurality of solid particles of a poorly water-soluble drug that possesses each of the following PSD parameters: D10≤0.15 μm, D50≤0.75 μm, and D90≤2.5. The liquid vehicle comprises, or consists essentially of, or consists of, one or more surfactant(s), one or more salt(s), and water. In preferred embodiments, the order of addition vis-à-vis the vehicle components is combining water and salt followed by adding particulate drug and surfactant, in no particular order or simultaneously. The method comprises the steps of: (i) providing a slurry of a starting population of the particulate drug dispersed in the liquid phase. The starting particulate drug population comprises, or consists essentially of, or consists of, a plurality of solid particles of the poorly water soluble drug that possesses one or more PSD parameter(s) of: D10>0.15 μm; D50>0.75 μm; and D90>2.5 μm; and (ii) subjecting the slurry to bead milling and/or high-pressure homogenization, and thereby simultaneously: (a) reducing the size of the starting population of particulate drug such that the population of solid particles thereof possesses each of the PSD parameters: D10<0.15 μm, D50<0.75 μm, and D90≤2.5, and (b) making the suspension of the size reduced particulate drug dispersed in the liquid vehicle.

In some embodiments, the size-reduced particulate drug population comprises, or consists essentially of, or consists of, a plurality of solid particles of a poorly water-soluble drug that possesses PSD parameters (i), (ii), and/or (iii), where: (i) is any one of D10≤5.0 μm, D10≤4.75 μm, D10≤4.5 μm, D10≤4.25 μm, D10≤4.0 μm, D10≤3.75 μm, D10≤3.5 μm, D10≤3.25 μm, D10≤3.0 μm, D10≤2.75 μm, D10≤2.5 μm, D10≤2.25 μm, D10≤2.0 μm, D10≤1.75 μm, D10≤1.5 μm, D10≤1.25 μm, D10≤1.0 μm, D10≤750 nm, D10≤500 nm, D10≤250 nm, D10≤100 nm, D10≤75 nm, D10≤50 nm, D10≤25 nm, D10≤10 nm; or D10≤1 nm; and (ii) is any one of D50≤15.0 μm, D50≤14 μm, D50≤13 μm, D50≤12 μm, D50≤11 μm, D50≤10 μm, D50≤9 μm, D50≤8 μm, D50≤7 μm, D50≤6 μm, D50≤5.0 μm, D50≤4.75 μm, D50≤ 4.5 μm, D50≤4.25 μm, D50≤4.0 μm, D50≤3.75 μm, D50≤3.5 μm, D50≤3.25 μm, D50≤3.0 μm, D50≤2.75 μm, D50≤2.5 μm, D50≤2.25 μm, D50≤2.0 μm, D50≤1.75 μm, D50≤1.5 μm, D50≤1.25 μm, D50≤1.0 μm, D50≤750 nm, D50≤500 nm, D50≤250 nm, or D50≤100 nm; and (iii) is any one of D90≤50 μm, D90≤40 μm, D90≤30 μm, D90≤25 μm, D90≤22.5 μm, D90≤20 μm, D90≤17.5 μm, D90≤15.0 μm, D90≤14 μm, D90≤13 μm, D90≤12 μm, D90≤11 μm, D90≤10 μm, D90≤9 μm, D90≤8 μm, D90≤7 μm, D90≤6 μm, D90≤5.0 μm, D90≤4.75 μm, D90≤4.5 μm, D90≤4.25 μm, D90≤4.0 μm, D90≤3.75 μm, D90≤3.5 μm, D90≤3.25 μm, D90≤3.0 μm, D90≤2.75 μm, D90≤2.5 μm, D90≤2.25 μm, D90≤2.0 μm, D90≤1.75 μm, D90≤1.5 μm, D90≤1.25 μm, D90≤1.0 μm, D90≤750 nm, D90≤500 nm, D90≤250 nm, or D90≤100 nm.

In some embodiments, the starting particulate drug population comprises, or consists essentially of, or consists of, a plurality of solid particles of a poorly water-soluble drug that possesses PSD parameters (i), (ii), and/or (iii), where: (i) is any one of D10>5.0 μm, D10>4.75 μm, D10>4.5 μm, D10>4.25 μm, D10>4.0 μm, D10>3.75 μm, D10>3.5 μm, D10>3.25 μm, D10>3.0 μm, D10>2.75 μm, D10>2.5 μm, D10>2.25 μm, D10>2.0 μm, D10>1.75 μm, D10>1.5 μm, D10>1.25 μm, D10>1.0 μm, D10>750 nm, D10>500 nm, D10>250 nm, D10>100 nm, D10>75 nm, D10>50 nm, D10>25 nm, D10>10 nm; or D10>1 nm; and (ii) is any one of D50>15.0 μm, D50>14 μm, D50>13 μm, D50>12 μm, D50>11 μm, D50>10 μm, D50>9 μm, D50>8 μm, D50>7 μm, D50>6 μm, D50>5.0 μm, D50>4.75 μm, D50>4.5 μm, D50>4.25 μm, D50>4.0 μm, D50>3.75 μm, D50>3.5 μm, D50>3.25 μm, D50>3.0 μm, D50>2.75 μm, D50>2.5 μm, D50>2.25 μm, D50>2.0 μm, D50>1.75 μm, D50>1.5 μm, D50>1.25 μm, D50>1.0 μm, D50>750 nm, D50>500 nm, D50>250 nm, or D50>100 nm; and (iii) is any one of D90>50 μm, D90>40 μm, D90>30 μm, D90>25 μm, D90>22.5 μm, D90>20 μm, D90>17.5 μm, D90>15.0 μm, D90>14 μm, D90>13 μm, D90>12 μm, D90>11 μm, D90>10 μm, D90>9 μm, D90>8 μm, D90>7 μm, D90>6 μm, D90>5.0 μm, D90>4.75 μm, D90>4.5 μm, D90>4.25 μm, D90>4.0 μm, D90>3.75 μm, D90>3.5 μm, D90>3.25 μm, D90>3.0 μm, D90>2.75 μm, D90>2.5 μm, D90>2.25 μm, D90>2.0 μm, D90>1.75 μm, D90>1.5 μm, D90>1.25 μm, D90>1.0 μm, D90>750 nm, D90>500 nm, D90>250 nm, or D90>100 nm.

As used herein, the term, “D90” means the particle diameter, in micrometers (μm), below which particles having 90% of the cumulative volume of all the particles are present. Similarly, the term, “D50” means the particle diameter, in micrometers (μm), below which particles having 50% of the cumulative volume of all the particles are present. Similarly, the term “D10” means the particle diameter, in micrometers (μm), below which particles having 10% of the cumulative volume of all the particles are present.

As used herein, the terms “poorly soluble in water” or “poorly water soluble” or the like means that that the drug or other compound modified by such term has a solubility in water of at or less than 10 mg/ml, 1 mg/ml, 100 μg/ml, 10 μg/ml, 1 μg/ml, 100 ng/ml, 10 ng/ml, 1 μg/ml, 100 fg/ml, 10 fg/ml, or 1 fg/ml, or in a range between of two of these values.

A wide variety of drugs that are poorly soluble in water may be used in the methods of the present invention. Preferably, such drugs are in a pure, or substantially pure, form. For example, purity levels for drugs useful in the methods of the present invention are at equal to or greater than 85% w/w, 87.5% w/w, 90% w/w, 92.5% w/w, 95% w/w, 96% w/w, 97% w/w, 98% w/w, 99% w/w, or 100% w/w, or in a range between any two of these values. As used herein, “poorly soluble in water” means that that the drug has a solubility in water of at or less than 10 mg/ml, 1 mg/ml, 100 μg/ml, 10 μg/ml, 1 μg/ml, 100 ng/ml, 10 ng/ml, 1 μg/ml, 100 fg/ml, 10 fg/ml, or 1 fg/ml, or in a range between of two of these values.

Poorly water soluble drugs and other compounds useful in the present invention may be of any class and/or any therapeutic effect, such as (without limitation), analgesic, antiinflammatory, anthelmintic, antiallergic, antiarrhythmic, antibiotic, anticoagulants, antidepressants, antidiabetic, antiepileptic, antihistamine, antihypertensive, antimuscarinic, antimycobacterial, antineoplastic, immunosuppressant, antithyroid, antiviral, anxiolytic (hypnotics and neuroleptics), astringent, beta-adrenoceptor agonist or antagonist, cardiac inotropic, steroid, corticosteroids, glucocorticosteroid, cough suppressant, expectorant, mucolytic, diuretic, dopaminergic (antiparkinson), haemostatic, lipid regulating, muscle relaxant, parasympathomimetic, sex hormones (including steroids, e.g., testosterone and estrogen), stimulant, sympathomimetic, thyroid agents, vasodilator, contrast imaging agents for x-ray and magnetic resonance imaging techniques, radiolabeled atoms, radiolabeled antibody, fluorescent dye, fluorescent protein or peptide, radiolabeled protein or peptid. A full listing and description of these drugs and a full listing of species within each class can be found in Martindale: The Complete Drug Reference (40th ed.). London: Pharmaceutical Press. (2020) ISBN 978-0-85711-367-2, the disclosure of which is hereby incorporated herein by reference in its entirety. For purposes of the present invention, “other poorly water-soluble compounds” include compounds useful for imaging the bodies of animals, including mammals such as humans, such as dyes, radiolabeled chemicals and biological molecules, and the like.

Poorly water-soluble drugs or other compounds may be present in the vehicles of the disclosure in weight to volume proportions, of the overall vehicle, of 0.1% w/v, 0.2% w/v, 0.3% w/v, 0.4% w/v, 0.5% w/v, 0.6% w/v, 0.7% w/v, 0.8% w/v, 0.9% w/v, 1.0% w/v, 1.1% w/v, 1.2% w/v, 1.3% w/v, 1.4% w/v, 1.5% w/v, 1.6% w/v, 1.7% w/v, 1.8% w/v, 1.9% w/v, 2.0% w/v, 2.5% w/v, 3.0% w/v, 3.5% w/v, 4.0% w/v, 4.5% w/v, 5.0% w/v, 7.5% w/v, 10% w/v, 12.5% w/v, 15% w/v, 20% w/v, 25% w/v, 30% w/v, or in a ranges between any two of said proportions. The formulations may contain combinations of poorly water-soluble drugs or other compounds, in amounts that individually or in aggregate achieve(s) the stated weight to volume proportions.

Surfactants useful in the aqueous vehicles of the disclosure include, without limitation, sodium lauryl sulfate, docusate sodium, phosphatidylcholine, lecithin, betaines, tyloxapol, polyoxyethylene sorbitan esters, such as polysorbate 20, polysorbate 60, and polysorbate 80; polyethoxylated castor oils, such as cremaphor, polyethoxylated hydrogenated castor oils, such as HCO-40; and poloxamers. Such surfactants may be present in aqueous vehicles of the disclosure in weight to volume proportions, of the overall vehicle, of 0.001% w/v, 0.005% w/v, 0.01% w/v, 0.05% w/v, 0.1% w/v, 0.2% w/v, 0.3% w/v, 0.4% w/v, 0.5% w/v, 0.6% w/v, 0.7% w/v, 0.8% w/v, 0.9% w/v, 1.0% w/v, 1.1% w/v, 1.2% w/v, 1.3% w/v, 1.4% w/v, 1.5% w/v, 1.6% w/v, 1.7% w/v, 1.8% w/v, 1.9% w/v, 2.0% w/v, 2.5% w/v, 3.0% w/v, 3.5% w/v, 4.0% w/v, 4.5% w/v, 5.0% w/v, 7.5% w/v, 10% w/v, 12.5% w/v, 15% w/v, or in a ranges between any two of said proportions. The formulations may contain combinations of surfactants, in amounts that individually or in aggregate achieve(s) the stated weight to volume proportions.

Salts useful in the aqueous vehicles of the disclosure include, without limitation, calcium acetate, calcium chloride, calcium citrate, calcium diphosphate, calcium maleate, calcium mesylate, calcium nitrate, calcium nitrite, calcium phosphate, calcium sulfate, calcium tartrate, potassium acetate, potassium chloride, potassium citrate, potassium diphosphate, potassium maleate, potassium mesylate, potassium nitrate, potassium nitrite, potassium phosphate, potassium sulfate, potassium tartrate, sodium acetate, sodium chloride, sodium citrate, sodium diphosphate, sodium maleate, sodium mesylate, sodium nitrate, sodium nitrite, sodium phosphate, sodium sulfate, and sodium tartrate. Such salts may be present in aqueous vehicles of the disclosure in weight to volume proportions, of the overall vehicle, of 0.001% w/v, 0.005% w/v, 0.01% w/v, 0.05% w/v, 0.1% w/v, 0.2% w/v, 0.3% w/v, 0.4% w/v, 0.5% w/v, 0.6% w/v, 0.7% w/v, 0.8% w/v, 0.9% w/v, 1.0% w/v, 1.1% w/v, 1.2% w/v, 1.3% w/v, 1.4% w/v, 1.5% w/v, 1.6% w/v, 1.7% w/v, 1.8% w/v, 1.9% w/v, 2.0% w/v, 2.5% w/v, 3.0% w/v, 3.5% w/v, 4.0% w/v, 4.5% w/v, 5.0% w/V, 7.5% w/v, 10% w/v, 12.5% w/v, 15% w/v, or in a ranges between any two of said proportions. The vehicles may contain combinations of salts, in amounts that individually or in aggregate achieve(s) the stated weight to volume proportions.

Buffers and pH adjusting agents may be useful in the aqueous vehicles of the disclosure. Buffers useful in formulations of the disclosure include, without limitation, acetic acid, sodium acetate, benzoic acid, sodium benzoate, boric acid, sodium borate, citric acid, sodium citrate, sodium phosphate, monobasic sodium phosphate, dibasic sodium phosphate, potassium phosphate, monobasic potassium phosphate, dibasic potassium phosphate, sodium acetate, lactic acid, a tartaric acid, sodium tartrate, sodium bicarbonate, sodium carbonate, tris(hydroxymethyl)aminomethane (“TRIS”), or a combination thereof. Acidic pH adjusting agents useful in vehicles of the disclosure include, without limitation, fumaric acid, formic acid, acetic acid, trichloroacetic acid, benzoic acid, oxalic acid, hydrofluoric acid, hydrogen sulfide, nitrous acid, sulfurous acid, phosphoric acid, and combinations thereof. Alkaline pH adjusting useful in vehicles of the disclosure include, without limitation, sodium hydroxide, potassium hydroxide, calcium hydroxide, ammonium carbonate, ammonium hydroxide, ethanolamine, and trolamine. Buffers and/or pH adjusting agents may be present in vehicles of the disclosure in amounts, alone or together, that are sufficient to cause the vehicles to have a pH of from 1 to 11, for example pH 1, pH 1.1, pH 1.2, pH 1.3, pH 1.4, t pH 1.5, pH 1.1, pH 1.7, pH 1.8, pH 1.9, pH 2, pH 2.1, pH 2.2, pH 2.3, pH 2.4, t pH 2.5, pH 2.2, pH 2.7, pH 2.8, pH 2.9, pH 3, pH 3.1, pH 3.2, pH 3.3, pH 3.4, t pH 3.5, pH 3.3, pH 3.7, pH 3.8, pH 3.9, pH 4, pH 4.1, pH 4.2, pH 4.3, pH 4.4, t pH 4.5, pH 4.4, pH 4.7, pH 4.8, pH 4.9, pH 5, pH 5.1, pH 5.2, pH 5.3, pH 5.4, t pH 5.5, pH 5.5, pH 5.7, pH 5.8, pH 5.9, pH 6, pH 6.1, pH 6.2, pH 6.3, pH 6.4, t pH 6.5, pH 6.6, pH 6.7, pH 6.8, pH 6.9, pH 7, pH 7.1, pH 7.2, pH 7.3, pH 7.4, pH 7.5, pH 7.6, pH 7.7, pH 7.8, pH 7.9, pH 8, pH 8.1, pH 8.2, pH 8.3, pH 8.4, pH 8.5, pH 8.6, pH 8.7, pH 8.8, pH 8.9, pH 9, pH 9.1, pH 9.2, pH 9.3, pH 9.4, pH 9.5, pH 9.6, pH 9.7, pH 9.8, pH 9.9, pH 10, pH 10.1, pH 10.2, pH 10.3, pH 10.4, pH 10.5, pH 10.6, pH 10.7, pH 10.8, pH 10.9, pH 11, as well as in a range between any two such pH values.

Polymers may be useful in the aqueous vehicles of the disclosure. Non-ionic polymers useful in vehicles of the disclosure include, without limitation, polyethylene glycol, hydroxyethyl cellulose, hydroxypropylmethylcellulose (e.g., 4000 MPA), methyl cellulose, carboxymethyl cellulose, polyvinyl pyrrolidone, and polyvinyl alcohol. Ionic polymers useful in vehicles of the disclosure include, without limitation, polyacrylates (e.g., carbopols and carbomers), alginates, chitosans, hyaluronic acid, and xanthan gum. Such ionic and/or nonionic polymers may be present in vehicles of the disclosure in weight to volume proportions of the overall vehicle of 0.001% w/v, 0.005% w/v, 0.01% w/v, 0.05% w/v, 0.1% w/v, 0.25% w/v, 0.5% w/v, 0.75% w/v, 1.0% w/v, 1.25% w/v, 1.5% w/v, 1.75% w/v, 2.0% w/v, 2.25% w/v, 2.5% w/v, 2.75% w/v, 3.0% w/v, 3.25% w/v, 3.5% w/v 3.75% w/v, 4.0% w/v, 4.25% w/v, 4.5% w/v, 4.75% w/v, or 5.0% w/v, as well as in a range between any two of said polymer proportions. The vehicles may contain combinations of polymers, in amounts that individually or in aggregate achieve(s) the stated polymer proportions.

Tonicity agents useful in vehicles of the disclosure include, without limitation, mannitol, sorbitol, xylitol, erythritol, lactitol, maltitol, isomalt, glycerol, propylene glycol, and combinations thereof. The vehicles may contain tonicity agent in weight to volume proportions of the overall vehicle of 0.001% w/v, 0.005% w/v, 0.01% w/v, 0.05% w/v, 0.1% w/v, 0.2% w/v, 0.3% w/v, 0.4% w/v, 0.5% w/v, 0.6% w/v, 0.7% w/v, 0.8% w/v, 0.9% w/v, 1.0% w/v, 1.1% w/v, 1.2% w/v, 1.3% w/v, 1.4% w/v, 1.5% w/v, 1.6% w/v, 1.7% w/v, 1.8% w/v, 1.9% w/v, 2.0% w/v, 2.1% w/v, 2.2% w/v, 2.3% w/v, 2.4% w/v, 2.5% w/v, 2.6% w/v, 2.7% w/v, 2.8% w/v, 2.9% w/v, 3.0% w/v, 3.3% w/v, 3.2% w/v, 3.3% w/v, 3.4% w/v, 3.5% w/v, 3.6% w/v, 3.7% w/v, 3.8% w/v, 3.9% w/v, 4.0% w/v, 4.1% w/v, 4.2% w/v, 4.3% w/v, 4.4% w/v, 4.5% w/v, 4.6% w/v, 4.7% w/v, 4.8% w/v, 4.9% w/v, 5.0% w/v, or in a range between any two of said tonicity agent proportions. The vehicles may contain combinations of tonicity agent, in amounts that individually or in aggregate achieve(s) the stated tonicity weight to volume proportions.

Preservatives useful in vehicles of the disclosure include, without limitation, dibutylhydroxytoluene, benzalkonium chloride, benzyl alcohol, borates, parabens, cresols, benzoic acid, phenol, sorbic acid, benzethonium chloride, sodium chlorite and combinations thereof. The vehicles may contain preservative in weight to volume proportions of the overall vehicle of 0.001% w/v, 0.005% w/v, 0.01% w/v, 0.05% w/v, 0.1% w/v, 0.25% w/v, 0.5% w/v, 0.75% w/v, 1.0% w/v, 1.25% w/v, 1.5% w/v, 1.75% w/v, 2.0% w/v, 2.25% w/v, 2.5% w/v, 2.75% w/v, 3.0% w/v, 3.25% w/v, 3.5% w/v 3.75% w/v, 4.0% w/v, 4.25% w/v, 4.5% w/v, 4.75% w/v, and 5.0% w/v, or in a range between any two of said preservative proportions. The vehicles may contain combinations of preservatives, in amounts that individually or in aggregate achieve(s) the stated weight to volume proportions.

Bead milling apparatus useful as size-reducing techniques for particulate drugs of the disclosure can, without limitation, consist of a hollow cylindrical shell rotating about its axis. The inner surface of the cylindrical shell is usually lined with an abrasion-resistant material such as manganese steel or rubber. Less wear takes place in rubber lined mills. The length of the mill is typically approximately equal to its diameter. The axis of the cylindrical shell may be either horizontal or at a small angle to the horizontal. In operation: (i) the cylindrical shell is partially filled with beads (i.e., the grinding media) and particulate drug (i.e., material to be size-reduced); and (ii) a cascading effect of the grinding media and particulate drug is established inside the rotating cylindrical shell, which reduces the size of the particulate drug.

Beads useful as grinding media in the ball-milling technique for size-reducing particulate drug of the disclosure can be made of, without limitation, agate, alumina, ceramic, flint, stainless steel, steel chrome, tungsten carbide, zirconia, or mixtures thereof. The size of such beads can be, without limitation, 1 mm, 2 mm, to 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, or mixtures thereof. Ball-milling operations are conducted for any amount of time appropriate to attain the desired PSD, at any temperature that the vehicle is liquid and the particulate drug targeted for size reduction is stable. For instance 15 minutes, 30 minutes, 45 minutes, 1 hour, 2 hours, 3 hours, 4 hours, 5 hours, 6 hours, 7 hours, 8 hours, 9 hours, 2 hours, 10, hours, 11 hours, 12 hours, 13 hours, 14 hr, 15 hours, 16 hours, 17 hours, 18 hours, 19 hours, 20 hours, 21 hours, 22 hours, 23 hours, 24 hours, 30 hours, 36 hours, 40 hours, hours, 44 hours, 48 hours, 52 hours, 56 hours, 60 hours, 64 hours, 68 hours, 72 hours, 4 days, 5 days, 6 days, 7 days, 10 days, 12 days, or 14 days, or in a range between any two of said ball-milling times. Also for instance, 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 35° C., 40° C., 42° C., 45° C., 50° C., 55° C., 60° C., 65° C., 70° C., 75° C., 80° C., 85° C., 90° C., or 95° C., or in a range between any two of said preservative temperatures.

High pressure homogenization apparatus useful a size-reducing techniques for particulate drugs of the disclosure can, without limitation, consist of an air motor connected to a hydraulic pump which circulates the fluid vehicle fluid. The vehicle stream is propelled at high pressures through an interaction chamber which has fixed microchannels that focus the vehicle stream and accelerate it to a high velocity. Within such chamber the vehicle is subjected to intense pressures shear, impact, and cavitation, all of which contribute to size reduction of the particulate drug. After such processing, the vehicle stream is passed through a heat exchanger coil and can be collected or recirculated through the apparatus. The heat exchanger and interaction chamber can be externally cooled with refrigerated circulating water or other coolant. Pressures in the interaction chamber can be 1,000 PSI, 2,000 PSI, 3,000 PSI, 4,000 PSI, 5,000 PSI, 6,000 PSI, 7,000 PSI, 8,000 PSI, 9,000 PSI, 10,000 PSI, 11,000 PSI, 12,000 PSI, 13,000 PSI, 14,000 PSI, 15,000 PSI, 16,000 PSI, 17,000 PSI, 18,000 PSI, 19,000 PSI, 20,000 PSI, 21,000 PSI, 22,000 PSI, 23,000 PSI, 24,000 PSI, 25,000 PSI, 26,000 PSI, 27,000 PSI, 28,000 PSI, 29,000 PSI, 30,000 PSI, 32,500 PSI, 35,000 PSI, 37,500 PSI, 40,000 PSI, 42,500 PSI, 45,000 PSI, 47,500 PSI, and 50,000 PSI.

High-pressure homogenizer mediated milling operations for particulate drug are conducted for any number of discrete, repeated passes through the high-pressure homogenizer apparatus or for any amount of recirculation times appropriate to attain the desired PSD, at any temperature that the vehicle is liquid and the particulate drug targeted for size reduction is stable. For instance 1 pass, 2 passes, 3 passes, 4 passes, 5 passes, 6 passes, 7 passes, 8 passes, 9 passes, 2 passes, 10, passes, 11 passes, 12 passes, 13 passes, 14 hr, 15 passes, 16 passes, 17 passes, 18 passes, 19 passes, 20 passes, 25 passes, 30 passes, 35 passes, 40 passes, 45 passes, 50 passes, 75 passes, 100 passes, 150 passes, 200 passes, 250 passes, 300 passes, 400 passes, or 500 passes, or in a range between any two of said number of discrete, repeated (if more than one) passes. For instance recirculation times of 15 minutes, 30 minutes, 45 minutes, 1 hour, 2 hours, 3 hours, 4 hours, 5 hours, 6 hours, 7 hours, 8 hours, 9 hours, 2 hours, 10, hours, 11 hours, 12 hours, 13 hours, 14 hr, 15 hours, 16 hours, 17 hours, 18 hours, 19 hours, 20 hours, 21 hours, 22 hours, 23 hours, 24 hours, 30 hours, 36 hours, 40 hours, hours, 44 hours, 48 hours, 52 hours, 56 hours, 60 hours, 64 hours, 68 hours, 72 hours, 4 days, 5 days, 6 days, 7 days, 10 days, 12 days, or 14 days, or in a range between any two of said recirculation times. Also for instance, 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 35° C., 40° C., 42° C., 45° C., 50° C., 55° C., 60° C., 65° C., 70° C., 75° C., 80° C., 85° C., 90° C., or 95° C., or in a range between any two of said temperatures.

### EXAMPLES

Aspects of embodiments of the present disclosure may be further understood in light of the following examples, which should not be construed as limiting in any way. Objectives of the PSD studies of the present disclosure were to evaluate the extent of a population of particles of a poorly water soluble drug or other compound in variety of liquid, aqueous vehicles.

### Example 1

API Particle size reduction. The API, prednisolone acetate, of the present invention is destined for inclusion in ophthalmic formulation and characterized by a target PSD of: D10≤0.15 μM, D50≤0.75 μM, D90≤2.5 μM. Unprocessed prednisolone acetate direct from synthesis had a PSD of: D10=1 μM, D50=2.5 μM, D90=6 μM. Further, it was immiscible with water, having the unflagging property of floating on the surface of water. Addition of surfactant (e.g. polysorbate) to aqueous solution was required to form slurries of prednisolone acetate suitable for use as vehicles in particle size reduction experiments that employed bead milling and high-pressure homogenization.

Several particle size reduction techniques and processes were investigated, namely: (i) ball milling, and (ii) high-pressure homogenization with apparatuses by microfluidizer, GEA, and emulsiflex. However, none of these particle size reduction techniques achieved target distribution in three component particle size reduction vehicles that consisted of prednisolone acetate, polysorbate, and water. Therefore, four and five component particle size reduction vehicles were the subject of further experimentation; and certain of such vehicles surprisingly and unexpectedly attained target prednisolone acetate PSD.

1.1 Ball Milling Trials. In one set of experiments, prednisolone acetate was formulated into several processing vehicles for ball mill trials, and for each such vehicle prednisolone acetate PSD was determined. Table 1.1 reports the composition of each vehicle, together with the PSD results for each. All ball mill trials were conducted with 3 mm zirconium beads. As can be seen, vehicles lacking salt did not attain target prednisolone PSD; whereas vehicles having salt did.

TABLE 1.1
PSD Results - Ball Milling Trials
Vehicle Compositions, Ball Mill Occupancies (“BMO”), Mill Times, and PSD results

| Vehicle | | BMO | PSD (μM) | | | PSD Target |
|---|---|---|---|---|---|---|
| No. | Vehicle Composition | Mill Time | D10 | D50 | D90 | Attainment |
| 24 | 10% w/w prednisolone acetate | 90% BMO | 1.2 | 1.9 | 3.3 | No |
| | 0.5% w/w polysorbate 80 | 48 hrs | | | | |
| | QS to 100% w/w water | | | | | |
| 27 | 10% w/w prednisolone acetate | 90% BMO | 0.1 | 1.2 | 3.1 | No |
| | 0.5% w/w polysorbate 80 | 48 hrs | | | | |
| | QS to 100% w/w water | | | | | |
| 29 | 10% w/w prednisolone acetate | 70% BMO | 0.6 | 7.7 | 24.9 | No |
| | 0.5% w/w polysorbate 80 | 48 hrs | | | | |
| | QS to 100% w/w water | | | | | |
| 33 | 10% w/w prednisolone acetate | 90% BMO | 0.85 | 1.6 | 3.1 | No |
| | 0.5% w/w polysorbate 80 | 48 hrs | | | | |
| | QS to 100% w/w water | | | | | |
| 37 | 10% w/w prednisolone acetate | 90% BMO | 1.3 | 2.1 | 3.9 | No |
| | 0.5% w/w polysorbate 80 | 48 hrs | | | | |
| | 1.2% w/w HPMC | | | | | |
| | QS to 100% w/w water | | | | | |
| 49 | 5% w/w prednisolone acetate | 90% BMO | 0.1 | 1.3 | 3.0 | No |
| | 0.3% w/w polysorbate 80 | 48 hrs | | | | |
| | QS to 100% w/w water | | | | | |
| 50 | 10% w/w prednisolone acetate | 90% BMO | 0.8 | 0.15 | 0.3 | Yes |
| | 0.5% w/w polysorbate 80 | 48 hrs | | | | |
| | 0.4% w/w HPMC | | | | | |
| | 1% w/w sodium citrate | | | | | |
| | QS to 100% w/w water | | | | | |
| 51 | 10% w/w prednisolone acetate | 90% BMO | 0.8 | 0.15 | 1.0 | Yes |
| | 0.5% w/w polysorbate 80 | 24 hrs | | | | |
| | 0.4% w/w HPMC | | | | | |
| | 1% w/w sodium citrate | | | | | |
| | QS to 100% w/w water | | | | | |
| 54 | 10% w/w prednisolone acetate | 90% BMO | 0.8 | 0.16 | 1.9 | Yes |
| | 0.5% w/w polysorbate 80 | 12 hrs | | | | |
| | 0.4% w/w HPMC | | | | | |
| | 1% w/w sodium citrate | | | | | |
| | QS to 100% w/w water | | | | | |
| 56 | 10% w/w prednisolone acetate | 90% BMO | 1.2 | 2.1 | 3.7 | No |
| | 0.5% w/w polysorbate 80 | 24 hrs | | | | |
| | 0.08% w/w BKC | | | | | |
| | QS to 100% w/w water | | | | | |
| 57 | 10% w/w prednisolone acetate | 90% BMO | 0.1 | 0.16 | 1.7 | Yes |
| | 0.5% w/w polysorbate 80 | 24 hrs | | | | |
| | 3% w/w sodium citrate | | | | | |
| | QS to 100% w/w | | | | | |
| 64 | 10% w/w prednisolone acetate | 90% BMO | 0.1 | 0.22 | 2.3 | Yes |
| | 0.5% w/w polysorbate 80 | 24 hrs | | | | |
| | 3% w/w sodium citrate | | | | | |
| | QS to 100% w/w water | | | | | |

^{♦}HPMC = hydroxypropylmethylcellulose

The vehicles employed in the ball milling experiments were made as follows. The quantity of tween 80 required for the planned vehicle and its batch size was weighed and added to a beaker, to which water was added in the amount required to achieve 60% w/w of the planned batch size. The polysorbate 80 and water were mixed until a clear solution was formed. To the clear, aqueous solution of polysorbate 80, the balance of the excipients for the planned vehicle were added, in the quantities required for the planned batch size and, if needed, mixed until a clear polysorbate-excipient solution was formed. The quantity of prednisolone acetate required for the planned vehicle and batch size was added, and then mixed into, the polysorbate-excipient solution until a uniform slurry was formed. To the uniform slurry, the quantity of water sufficient to achieve 100% of the planned vehicle and batch size was added. As needed, finished vehicles (e.g., vehicles 24 and 49) de-agglomerated by subjugation to high shear homogenization for 30 minutes. 35 g samples of each finished (and de-agglomerated if needed) vehicle were transferred to a 125 cc jar and therein subjected to ball milling with 3 mm zirconium beads, in a Planetary Ball Mill 100 apparatus, at 300 RPM and for 12 hours, 24 hours, or 48 hours.

1.2 High-Pressure Homogenization Trials-Avestin Emulsiflex. In another set of experiments, API prednisolone acetate was formulated into several processing vehicles for high pressure homogenization trials in the Avestin Emulsiflex, and for each such vehicle prednisolone acetate PSD was determined. Table 1.2 reports the composition of each vehicle, the high-pressure homogenization parameters, and PSD results. As can be seen, vehicles lacking salt did not attain target prednisolone PSD; whereas vehicles having salt did.

TABLE 1.2
PSD Results - Avestin Emulsiflex Trials
Vehicle Compositions, High Pressure Homogenizer Pressure and Passes, and PSD results

| Vehicle | | HPH PSI | PSD (μM) | | | PSD Target |
|---|---|---|---|---|---|---|
| No. | Vehicle Composition | HPH Passes | D10 | D50 | D90 | Attainment |
| 44 | 5% w/w prednisolone acetate | 20,000 PSI | 0.1 | 1.3 | 2.8 | No |
| | 0.25% w/w polysorbate 80 | 50 Passes | | | | |
| | QS to 100% w/w water | | | | | |
| 45 | 5% w/w prednisolone acetate | 22,500 PSI | 0.3 | 1.6 | 3.3 | No |
| | 0.25% w/w polysorbate 80 | 50 Passes | | | | |
| | QS to 100% w/w water | | | | | |
| 53 | 5% w/w prednisolone acetate | 20,000 PSI | 0.1 | 0.67 | 2.13 | Yes |
| | 0.25% w/w polysorbate 80 | 50 Passes | | | | |
| | 0.24% w/w HPMC | | | | | |
| | 0.6% w/w sodium citrate | | | | | |
| | QS to 100% w/w water | | | | | |
| 77 | 5% w/w prednisolone acetate | 20,000 PSI | 0.1 | 0.60 | 2.1 | Yes |
| | 0.25% w/w polysorbate 80 | 50 Passes | | | | |
| | 1.5% w/w sodium citrate | | | | | |
| | QS to 100% w/w water | | | | | |

HPMC = hydroxypropylmethylcellulose PSI = pounds per square inch

1.3 High-Pressure Homogenization Trials-Microfluidics Microfluidizer. In another set of experiments, API prednisolone acetate was formulated into several processing vehicles for high pressure homogenization trials in the Microfluidics microfluidizer, and for each such vehicle prednisolone acetate PSD was determined. Table 1.3 reports the composition of each vehicle, the high-pressure homogenization parameters, and PSD results. As can be seen, vehicles lacking salt did not attain target prednisolone PSD; whereas vehicles having salt did.

TABLE 1.3
PSD Results - Microfluidics Microfluidizer Trials
Vehicle Compositions, High Pressure Homogenizer Pressure and Passes, and PSD results

| Vehicle | | HPH PSI | PSD (μM) | | | PSD Target |
|---|---|---|---|---|---|---|
| No. | Vehicle Composition | HPH Passes | D10 | D50 | D90 | Attainment |
| 02 | 10% w/w prednisolone acetate | 25,000 PSI | 1.4 | 2.5 | 4.4 | No |
| | 0.5% w/w polysorbate 80 | 150 passes | | | | |
| | QS to 100% w/w water | | | | | |
| 08 | 10% w/w prednisolone acetate | 27,000 PSI | 1.3 | 2.3 | 4.2 | No |
| | 0.5% w/w polysorbate 80 | 50 passes | | | | |
| | QS to 100% w/w water | | | | | |
| 55 | 10% w/w prednisolone acetate | 25,000 PSI | 0.08 | 0.21 | 1.10 | Yes |
| | 0.5% w/w polysorbate 80 | 150 passes | | | | |
| | 0.4% w/w HPMC | | | | | |
| | 0.9% w/w sodium citrate | | | | | |
| | QS to 100% w/w water | | | | | |
| 58 | 10% w/w prednisolone acetate | 25,000 PSI | 0.09 | 0.26 | 1.32 | Yes |
| | 0.5% w/w polysorbate 80 | 125 passes | | | | |
| | 0.4% w/w HPMC | | | | | |
| | 3.0% w/w sodium citrate | | | | | |
| | QS to 100% w/w water | | | | | |
| 60B | 10% w/w prednisolone acetate | 25,000 PSI | 0.09 | 0.47 | 1.90 | Yes |
| | 0.5% w/w polysorbate 80 | 125 passes | | | | |
| | 12% w/w sodium sulfate | | | | | |
| | QS to 100% w/w water | | | | | |
| 75 | 10% w/w prednisolone acetate | 25,000 PSI | 0.09 | 0.38 | 1.7 | Yes |
| | 0.5% w/w polysorbate 80 | 100 passes | | | | |
| | 0.4% w/w HPMC | | | | | |
| | 3.0% w/w Sodium citrate | | | | | |
| | QS to 100% w/w water | | | | | |
| 76 | 10% w/w prednisolone acetate | 25,000 PSI | 0.09 | 0.6 | 1.9 | Yes |
| | 0. % w/w polysorbate 80 | 60 passes | | | | |
| | 0.4% w/w HPMC | | | | | |
| | 3.0% w/w sodium citrate | | | | | |
| | QS to 100% w/w water | | | | | |
| 87 | 10% w/w prednisolone acetate | 25,000 PSI | 0.09 | 0.5 | 1.8 | Yes |
| | 0.5% w/w polysorbate 80 | 60 passes | | | | |
| | 0.24% w/w HPMC | | | | | |
| | 3.0% w/w sodium citrate | | | | | |
| | QS to 100% w/w water | | | | | |
| 90 | 10% w/w prednisolone acetate | 25,000 PSI | 0.09 | 0.40 | 1.7 | Yes |
| | 0.7% w/w tyloxapol | 100 passes | | | | |
| | 1.5% w/w sodium sulfate | | | | | |
| | QS to 100% w/w water | | | | | |
| 97 | 10% w/w prednisolone acetate | 25,000 PSI | 0.09 | 0.41 | 1.9 | Yes |
| | 0.5% w/w polysorbate 80 | 60 passes | | | | |
| | 0.25% w/w HPMC | | | | | |
| | 3.0% w/w sodium citrate | | | | | |
| | QS to 100% w/w water | | | | | |
| 99 | 20% w/w prednisolone acetate | 25,000 PSI | 0.09 | 0.41 | 2.0 | Yes |
| | 1.0% w/w polysorbate 80 | 60 passes | | | | |
| | 0.25% w/w HPMC | | | | | |
| | 6.0% w/w sodium citrate | | | | | |
| | QS to 100% w/w water | | | | | |
| 102 | 20% w/w prednisolone acetate | 25,000 PSI | 0.08 | 0.30 | 1.5 | Yes |
| | 1.0% w/w polysorbate 80 | 60 passes | | | | |
| | 0.5% w/w HPMC | | | | | |
| | 6.0% w/w sodium citrate | | | | | |
| | 4.0% w/w boric acid | | | | | |
| | QS to 100% w/w water | | | | | |
| 103 | 20% w/w prednisolone acetate | 25,000 PSI | 0.09 | 0.40 | 2.0 | Yes |
| | 1.0% w/w polysorbate 80 | 60 passes | | | | |
| | 0.5% w/w HPMC | | | | | |
| | 3.0% w/w sodium citrate | | | | | |
| | 4.0% boric acid | | | | | |
| | QS to 100% w/w water | | | | | |
| 104 | 20% w/w prednisolone acetate | 25,000 PSI | 0.09 | 0.65 | 2.3 | Yes |
| | 1.0% w/w polysorbate 80 | 60 passes | | | | |
| | 0.5% w/w HPMC | | | | | |
| | 1.5% w/w sodium citrate | | | | | |
| | 4.0% w/w boric acid | | | | | |
| | QS to 100% w/w water | | | | | |
| 105 | 20% w/w prednisolone acetate | 30,000 PSI | 0.09 | 0.40 | 1.7 | Yes |
| | 1.0% w/w polysorbate 80 | 45 passes | | | | |
| | 0.5% w/w HPMC | | | | | |
| | 1.5% w/w sodium citrate | | | | | |
| | 4.0% w/w boric acid | | | | | |
| | QS to 100% w/w water | | | | | |

HPMC = hydroxypropylmethylcellulose PSI = pounds per square inch

1.4 High-Pressure Homogenization Trials-GEA Microfluidizer. In another set of experiments, API prednisolone acetate was formulated into several processing vehicles for high pressure homogenization trials in the GEA microfluidizer, and for each such vehicle prednisolone acetate PSD was determined. Table 1.4 reports the composition of each vehicle, the high-pressure homogenization parameters, and PSD results. As can be seen, vehicles lacking salt did not attain target prednisolone PSD; whereas vehicles having salt did.

TABLE 1.4
PSD Results - GEA Microfluidizer Trials
Vehicle Compositions, High Pressure Homogenizer Pressure and Passes, and PSD results

| Vehicle | | HPH PSI | PSD (μM) | | | PSD Target |
|---|---|---|---|---|---|---|
| No. | Vehicle Composition | HPH Passes | D10 | D50 | D90 | Attainment |
| 21 | 10% w/w prednisolone acetate | 17,500 PSI | 0.543 | 3.15 | 6.0 | No |
| | 0.5% w/w polysorbate 80 | 120 passes | | | | |
| | QS to 100% w/w water | | | | | |
| 22 | 10% w/w prednisolone acetate | 17,500 PSI | 1.5 | 3.1 | 5.76 | No |
| | 0.5% w/w polysorbate 80 | 150 passes | | | | |
| | QS to 100% w/w water | | | | | |
| 62 | 10% w/w prednisolone acetate | 17,500 PSI | 0.09 | 0.40 | 1.44 | Yes |
| | 0.5% w/w polysorbate 80 | 150 passes | | | | |
| | 0.4% HPMC | | | | | |
| | 3.0% w/w sodium citrate | | | | | |
| | QS to 100% w/w water | | | | | |
| 83 | 5% w/w prednisolone acetate | 17,500 PSI | 0.09 | 0.65 | 1.9 | Yes |
| | 0.25% w/w polysorbate 80 | 120 passes | | | | |
| | 0.13% HPMC 4000 | | | | | |
| | 1.5% w/w sodium citrate | | | | | |
| | QS to 100% w/w water | | | | | |

HPMC = hydroxypropylmethylcellulose PSI = pounds per square inch

1.5 PSD determination by light diffraction. All PSD reported in Tables 1.1-1.4 were determined by light diffraction (“LD”) analysis using a Mastersizer 2000. All LD samples were prepared by pipetting 1.5 ml thereof into a test tube and vortexing for 1 minute and then sonicated for 10 second to remove any trapped air bubbles. Separately, 450 ml dispersant (Milli-Q water) was added to a cell assembly for the Mastersizer and sonicated for another 5 minute with stirring. Proper cell alignment within the Mastersizer 2000 was assured and then the background (blue and red light) was measured. Then test sample was added to the cell assembly, dropwise until the obscuration was in the range of five percent (5%) to ten percent (10%) and the PSD parameters D10, D50, and D90 were recorded.

TABLE 1.5
Mastersizer 2000 Parameters for PSD Determination

| Parameter | Value | |
|---|---|---|
| Detector Range | 0.02 μm-2000 μm | |
| Laser Intensity | More than 70.0% | |
| Sample unit | Hydro 2000 MU (A) | |
| Particle type | Non spherical | |
| Refractive index | 1.570 | |
| Absorption index | 0.01 | |
| Particle density | 1.05 | gm/cm³ |
| Dispersant | Water | |
| Background measurement | 2 | second |
| Sample measurement | 2 | second |
| Delay between measurement | 0 | second |
| Premeasurement delay | 0 | second |
| Number of measurements | 5 | |
| Obscuration Lower limit | 5% | |
| Obscuration Upper limit | 10% | |
| Enable obscuration filtering | Yes | |
| Stirrer speed | 2000 | rpm |

| | |
|---|---|
| Ultrasonic displacement | 5 Second @ 10% |
| Analysis | General purpose |
| Use the sample mode | Yes |
| Result | Volume Distribution |
| Averaging Enabled | Yes |
| Sample weight | RLD = 1.5 mL Generic = 1.5 mL |

1.7 Vehicle Preparation. The vehicles employed in the high-pressure homogenization and ball milling experiments were made as follows. The quantity of tween 80 required for the planned vehicle and its batch size was weighed and added to a beaker, to which water was added in the amount required to achieve 60% w/w of the planned batch size. The polysorbate 80 and water were mixed until a clear solution was formed. To the clear, aqueous solution of polysorbate 80, the balance of the excipients for the planned vehicle were added, in the quantities required for the planned batch size and, if needed, mixed until a clear polysorbate-excipient solution was formed. The quantity of prednisolone acetate required for the planned vehicle and batch size was added, and then mixed into, the polysorbate-excipient solution until a uniform slurry was formed. To the uniform slurry, the quantity of water sufficient to achieve 100% of the planned vehicle and batch size was added. As needed, finished vehicles (e.g., vehicles 24 and 49) de-agglomerated by subjugation to high shear homogenization for 30 minutes. 35 g samples of each finished vehicle (de-agglomerated if needed) were subjected to high-pressure homogenization conditions specified in Tables 1.2-1.4.

While the foregoing specification teaches the principles of the present invention, with examples provided for the purpose of illustration, it will be understood that the practice of the invention encompasses all variations, adaptations and/or modifications as come within the scope of the following claims and their equivalents.

What is claimed is:

1. A method for making a microfine suspension consisting essentially of a size-reduced population of drug particles dispersed in a liquid vehicle the size-reduced population of drug particles possessing each of the PSD parameters: D10<0.15 μm, D50≤0.75 μm, and D90≤2.5 μm, wherein:
said drug is a steroid, or a pharmaceutically acceptable salt thereof,
said vehicle consists essentially of a surfactant, a salt, and water, and
said method consists essentially of the steps:
(i) providing a slurry of a starting population of the drug particles, dispersed in the vehicle and possessing at least one of the PSD parameters: D10>0.15 μm; D50>0.75 μm; and D90>2.5 μm, and
(ii) subjecting the slurry to bead milling and/or high-pressure homogenization and thereby concomitantly: (a) reducing in size of the starting population of drug particles so that it possesses each of the PSD parameters: D10≤0.15 μm, D50≤0.75 μm, and D90≤2.5 μm, and (b) making the suspension.

2. The method of claim 1, wherein the amount of:
particulate drug in the vehicle is from 1% w/v to 30% w/v,
surfactant in the vehicle is from 0.125% w/v to 1.5% w/v, and
salt in the vehicle is from 0.5% w/v to 25% w/v.

3. The method of claim 2, wherein:
the drug is a corticosteroid selected from the group consisting of budesonide, betamethasone, cortisone, dexamethasone, hydrocortisone, mometasone, prednisolone, prednisone, triamcinolone, and combinations thereof;
the surfactant is selected from the group consisting of sodium lauryl sulfate, docusate sodium, phosphatidylcholine, lecithin, betaines, tyloxapol, polyoxyethylene sorbitan ester, polyethoxylated castor oil, polyethoxylated hydrogenated castor oils, poloxamer, and combinations thereof; and
the salt is selected from the group consisting of calcium acetate, calcium chloride, calcium citrate, calcium diphosphate, calcium maleate, calcium mesylate, calcium nitrate, calcium nitrite, calcium phosphate, calcium sulfate, calcium tartrate, potassium acetate, potassium chloride, potassium citrate, potassium diphosphate, potassium maleate, potassium mesylate, potassium nitrate, potassium nitrite, potassium phosphate, potassium sulfate, potassium tartrate, sodium acetate, sodium chloride, sodium citrate, sodium diphosphate, sodium maleate, sodium mesylate, sodium nitrate, sodium nitrite, sodium phosphate, sodium sulfate, sodium tartrate, and combinations thereof.

4. The method of claim 3, wherein the drug is prednisolone, the surfactant is at least one of the polyoxyethylene sorbitan ester and the tyloxapol, and the salt is at least one of the sodium citrate and sodium sulfate.

5. The method of claim 4, wherein the slurry is subjected to the bead milling for 12 hours to 60 hours at from 4° C. to 50° C.; and wherein the beads are from 1 mm to 10 mm in diameter and of material selected from the group consisting of agate, alumina, ceramic, flint, stainless steel, steel chrome, tungsten carbide, and zirconia, and combinations thereof.

6. The method of claim 4, wherein the slurry is subjected to the high-pressure homogenization for 15 passes to 250 passes, at from 10,000 PSI to 50,000 PSI and from 4° C. to 50° C., and wherein the drug is prednisolone acetate.

7. The method of claim 5, wherein the beads are 3 mm in diameter and of the zirconium, and wherein the drug is prednisolone acetate.

8. A method for making a suspension consisting essentially of a size-reduced population of prednisolone acetate particles dispersed in a liquid vehicle, the size-reduced population of prednisolone acetate particles possessing each of the PSD parameters: D10≤0.15 μm, D50≤0.75 μm, and D90≤2.5, wherein:
said vehicle consists essentially of: from 0.125% w/v to 1.5% w/v of at least one of a polysorbate and a tyloxapol; from 0.5% w/v to 25% w/v of a salt that is selected from the group consisting of calcium acetate, calcium chloride, calcium citrate, calcium diphosphate, calcium maleate, calcium mesylate, calcium nitrate, calcium nitrite, calcium phosphate, calcium sulfate, calcium tartrate, potassium acetate, potassium chloride, potassium citrate, potassium diphosphate, potassium maleate, potassium mesylate, potassium nitrate, potassium nitrite, potassium phosphate, potassium sulfate, potassium tartrate, sodium acetate, sodium chloride, sodium citrate, sodium diphosphate, sodium maleate, sodium mesylate, sodium nitrate, sodium nitrite, sodium phosphate, sodium sulfate, sodium tartrate, and combinations thereof; and water,
the amount of prednisolone acetate in the vehicle is from 1% w/v to 30% w/v, and
said method consists essentially of the steps:
(i) providing a slurry of a starting population of prednisolone acetate particles, which are dispersed in the vehicle and possess at least one of the PSD parameters: D10>0.1 μm; D50>0.5 μm; and D90>2 μm, and
(ii) subjecting the slurry to bead milling and/or high-pressure homogenization and thereby concomitantly: (a) reducing the size of the starting population of prednisolone acetate particles so that it possesses each of the PSD parameters: D10≤0.15 μm, D50≤0.75 μm, and D90≤2.5, and D90≤2 μm; and (b) making the suspension.

9. The method of claim 8, wherein the slurry is subjected to the bead milling for 12 hours to 60 hours at from 4° C. to 50° C., and wherein the beads are from 1 mm to 10 mm in diameter and of a material selected from the group consisting of agate, alumina, ceramic, flint, stainless steel, steel chrome, tungsten carbide, and zirconia, and combinations thereof.

10. The method of claim 8, wherein the slurry is subjected to the high-pressure homogenization for 15 passes to 250 passes, at from 10,000 PSI to 50,000 PSI and 4° C. to 50° C.

11. The method of claim 9, wherein the beads are 3 mm in diameter and of the zirconium.
